(19) United States
(12) Patent Application Publication
(10) Pub. No.: US 2019/0309422 A1
(43) Pub. Date: Oct. 10, 2019

(54) Spin-On Metallization

(71) Applicant: Versum Materials US, LLC, Tempe, AZ (US)

(72) Inventors: Alan C. Cooper, Tempe, AZ (US); Sergei V. Ivanov, Tempe, AZ (US); Hongbo Li, Tempe, AZ (US); Ronald Martin Pearlstein, Tempe, AZ (US); Xinjian Lei, Tempe, AZ (US)

(73) Assignee: Versum Materials US, LLC, Tempe, AZ (US)

(21) Appl. No.: 16/365,109

(22) Filed: Mar. 26, 2019

Related U.S. Application Data
(60) Provisional application No. 62/653,753, filed on Apr. 6, 2018.

(51) Int. Cl. C23C 18/32; C23C 18/16; H01L 21/285; H01L 21/768; C09D 5/24; C09D 11/52; C09D 11/322; C09D 11/037

(52) U.S. Cl. CPC C23C 18/32; C23C 18/1642; C23C 18/1682; C23C 18/1692; C09D 11/037; H01L 21/76838; C09D 5/24; C09D 11/52; C09D 11/322; H01L 21/28556

(57) ABSTRACT

Described herein are the depositions of conductive metallic films on a surface which contains topography. The deposition uses a metallic precursor comprises a neutral (uncharged) metal compound in which the metal atom is in the zerovalent state and stabilized by ligands which are stable as uncharged, volatile species.

### CROSS REFERENCE TO RELATED APPLICATIONS

This application claims priority to U.S. provisional application 62/653,753 filed on Apr. 6, 2018, the entire contents of which is incorporated herein by reference thereto for all allowable purposes.

### BACKGROUND OF THE INVENTION

The present invention relates to fabrication processing techniques of semiconductor devices and related devices. In particular, it relates to techniques for performing film deposition using a metallic element containing compound as a liquid or as a solution in a suitable solvent.

There are a number of conventional ways to lay down conductive lines or vias in semiconductor devices. One way is to do physical vapor deposition involving physical processes such as evaporation or sputtering of a metal or alloy from a metallic target onto the surface of the semiconductor wafer through the application of heat, ion beam or other energy source. Chemical vapor deposition, wherein a metallic or metal halide precursor in the vapor phase is selectively decomposed or chemically reduced on the surface. A subset of chemical vapor deposition is atomic layer deposition where the metal precursor and reducing agent are sequentially exposed to the surface to grow the metallic film in a layer by layer manner. Other techniques commonly employed include electroplating, wherein the wafer is coated with an electrolyte and connected to a DC electric circuit with the substrate serving as the cathode. When current is passed, metal ions dissolved in the electrolyte are chemically reduced on the surface of the cathode. Other techniques known in the art include electroless deposition (autocatalytic deposition) wherein a mixture of metallic ions and chemical reducing agents dissolved in a solvent are contacted to the substrate. A chemical reaction catalyzed by the surface leads to the reaction of the reducing agent with the metal ions to form a reduced metallic coating.

Examples of interconnect metallization of the prior art: U.S. Pat. Nos. 6,048,445; 5,151,168, 5,674,787.

There are many challenges of the prior art. In particular, many of these techniques, and in particular physical vapor deposition, have significant challenges in completely filling high aspect ratio features (i.e. features that are much deeper than they are wide at the opening). The gas-phase processes are typically also incapable of completely filling re-entrant features (i.e. features that have a narrow opening but expand laterally below the surface). Incomplete filling can lead to spots of high resistivity and cause current fluctuations and also lead to localized heating or exacerbate electromigration.

Atomic Layer Deposition (ALD) can, in principle, fill complex high aspect ratio features, but in practice often leaves a seam where the deposit growing inwards from each side-wall merges. Such seams can likewise lead to undesired defects in the electrical performance of the interconnect circuits.

Electroplating requires that a seed layer be deposited, and as dimensions of the features get smaller as the technology progresses, this becomes increasingly difficult.

Another challenge of the prior art is achieving acceptable electrical conductivity of the interconnect circuit.

U.S. Pat. No. 8,232,647 describes one approach to dealing with so-called keyhole defect formation or seams in conventional metallization.

JP2012012647A2 (WO201163235) by Tokyo Electron discloses use of a spin track under inert atmosphere wherein a solvent borne metal complex is deposited on the surface. This patent focuses on aluminum containing precursor but also discloses that silver, gold or copper. There is no description of preferred on suitable complexes for this application nor the use of zerovalent metal complexes, their pre-agglomeration, preference for using liquid or low melting point complexes. The Aluminum compounds referenced were Al(III) hydrides and amine adducts thereof. Such compounds decompose by reductive elimination, i.e. the ligands themselves act as the reducing agent.

U.S. Pat. No. 6,852,626B1 by Applied Materials, also referenced in the above, discloses decomposition of a metallic complex, specifically Cu(I)hfac(tmvs), on the surface to deposit a metallic copper film. Copper metal is formed by disproportionation into Cu(II) and Cu(O).

U.S. Pat. No. 9,653,306B2 by JSR details the use of a zerovalent Co precursor along with a silicon precursor (a silane or halosilane) to form a self-aligned cobalt silicide thin film.

Maria Careri et al studied high-performance liquid chromatography of trinuclear ruthenium acetylido-carbonyl compounds in Journal of Chromatography, 634 (1993) 143-148.

Thus, the development of precursors is necessary and is needed for a high purity film with controlled grain boundaries which maximally fills the circuit paths.

### SUMMARY

Described herein are the depositions of conductive metallic films on a surface which contains topography. The present invention uses a neutral (uncharged) metal compound as the precursor in which the metal atom is in the zerovalent state and stabilized by ligands which are stable as uncharged, volatile species.

In order to create conductive paths on a surface which has been patterned with recesses in a semiconductor substrate; a liquid metallic precursor containing a metallic compound as a liquid or as a solution in a suitable solvent is applied to the surface. The pool of liquid may be spread on the surface under inert conditions in a known manner so that the recessed areas are filled with this liquid by capillary action, optionally with excess liquid retained on top of the surface by the surface tension of the liquid. The substrate is then subjected to heating that leads to evaporation of the optional solvent and some of the stabilizing ligands, leading to partial decomposition of the precursor to form agglomerated metallic clusters or nanoparticles that on further heating coalesce in the recesses while they release the bulk of the stabilizing ligands to leave a conductive metallic solid. In a preferred embodiment of this invention, the metallic solid partially or substantially fills the gaps or recesses in high-aspect-ratio or reentrant features initially present on the surface of the substrate, and thereby enabling gap-filling.

The metallic precursors best suited for this process comprises a neutral (uncharged) metal compound having a metal in zerovalent state and at least one neutral stabilizing ligand

which can be released as neutral molecules.

The neutral (uncharged) metal compound can be a liquid or a solid which is soluble at ambient temperature (defined as 15° C. to 25° C.), in a solvent selected from the group consisting of saturated linear, branched and cyclic hydrocarbons; or can be a solid that melts at a temperature below a decomposition temperature.

The metallic precursor comprises the neutral (uncharged) metal compound or the neutral (uncharged) metal compound with the solvent.

A liquid metallic precursor has a viscosity at ambient temperature between 0.5 cP and 20 cP, preferably between 1 cP and 10 cP, and more preferably between 2 cP and 5 cP.

Examples of suitable metals include but are not limited to cobalt, ruthenium, iridium, rhodium, iron, osmium, nickel, platinum, palladium, copper, silver, gold, and combinations thereof.

Suitable neutral stabilizing ligands include but are not limited to carbon monoxide (CO), nitric oxide (NO), dinitrogen (N₂), acetylene (C₂H₂), ethylene (C₂H₄), C₄-C₁₈ diene or C₄-C₁₈ cyclic diene, C₆-C₁₈ triene, C₈-C₁₈ tetraene, organoisocyanide RNC wherein R═C₁ to C₁₂ linear branched hydrocarbyl or halocarbyl radical; organic nitrile RCN wherein R═C₁ to C₁₂ hydrocarbyl or halocarbyl radical; organophosphine PR′₃ wherein R′═H, Cl, F, Br, or a C₁ to C₁₂ hydrocarbyl or halocarbyl radical; amine NRaRbRc wherein Ra, Rb and Rc can be independently selected from H or a C₁ to C₁₂ hydrocarbyl or halocarbyl radical where they may be connected to each other; organic ether with general formula R*OR** wherein R* and R** can be selected independently from C₁ to C₁₂ hydrocarbyl or halocarbyl radicals and may be connected to each other; and terminal or internal alkyne with general formula R₁CCR₂ where R₁ and R₂ can be independently selected from H, C₁ to C₁₂ linear, branched, cyclic or aromatic halocarbyl or hydrocarbyl radical, silyl or organosilyl radical (e.g. Si(CH₃)₃), SiCl₃), stannyl or organostannyl radical, and combinations thereof.

Suitable metallic precursor includes, but is not limited to

R¹Co₂(CO)₆, wherein R¹ is a linear or branched C₂ to C₁₀ alkyne, a linear or branched C₁ to C₁₀ alkoxy alkyne, a linear or branched C₁ to C₁₀ organoamino alkyne such as (tert-butylacetylene)dicobalt hexacarbonyl; [Co₂(CO)₆HC:::CC(CH₃)₃];
R¹CoFe(CO)₇, wherein R¹ is a linear or branched C₂ to Co₁₀ alkyne, a linear or branched C₁ to C₁₀ alkoxy alkyne, a linear or branched C₁ to C₁₀ organoamino alkyne;
R²CCo₃(CO)₉, wherein R² is selected from the group consisting of hydrogen, a linear or branched C₁ to C₁₀ alkyl, a linear or branched C₁ to C₁₀ alkoxy, Cl, Br, COOH, COOMe, COOEt;
R²CCo₂Mn(CO)₁₀, wherein R² is selected from the group consisting of hydrogen, a linear or branched C₁ to C₁₀ alkyl, a linear or branched C₁ to C₁₀ alkoxy, Cl, Br, COOH, COOMe, COOEt;
R³Co₄(CO)₁₂, wherein R³ is selected from a linear or branched C₁ to C₁₀ alkenylidene; and

R⁴Ru₃(CO)₁₁ wherein R⁴ is selected from a disubstituted alkyne (R^{#}CCR^{##}) wherein R# and R## can be selected independently from C₁ to C₁₂ linear, branched, cyclic or aromatic halocarbyl or hydrocarbyl radical, silyl or organosilyl radical (e.g. Si(CH₃)₃), SiCl₃), stannyl or organostannyl radical, and combinations thereof. Suitable example of metallic precursor includes, but is not limited to dicobalthexacarbonyltert-butylacetylene [Co2(CO)6HC:::CC(CH3)3], (1-decyne) tetracobalt dodecacarbonyl (Co₄(CO)₁₂(C₈H₁₇C:::CH)), (1,6-Heptadiyne) tetracobalt dodecacarbonyl, (2,2,6-Trimethyl-3-heptyne) dicobalt hexacarbonyl, (2,2-Dimethyl-3-octyne) dicobalt hexacarbonyl (CCTNBA), (2,2-Dimethyl-3-decyne) dicobalt hexacarbonyl, (2,2-Dimethyl-3-heptyne) dicobalt hexacarbonyl, (tert-butylmethylacetylene)dicobalt hexacarbonyl (CCTMA), trirutheniumdodecacarbonyl, (ethylbenzene)(1,3-butadiene)Ruthenium, (isopropyl-4-methyl-Benzene)(1,3-butadiene)ruthenium, 1,3,5-cycloheptatrienedicarbonylruthenium, 1,3-cyclohexadienetricarbonylruthenium, 2,3-dimethyl-1,3-butadienetricarbonylruthenium, 2,4-hexadienetricarbonylruthenium, 1,3-pentadienetricarbonylruthenium, (benzene)(1,3-butadiene)ruthenium, (benzene)(2,3-Dimethyl-1,3-butadiene)ruthenium, CO₂Ru(CO)₁₁, HCoRu₃(CO)₁₃, Ru₃(CO)₉(PPh₂(CH₂)₃Si(OEt)₃)₃, bis(benzene)chromium, bis(cyclooctadiene)nickel, bis(tri-tert-butylphosphine)platinum, bis(tri-tert-butylphosphine)palladium, and combinations thereof.

In another aspect, described herein is a method to deposit a conductive metallic film onto a substrate comprising:
a. providing the substrate with a surface containing topography;
b. providing the metallic precursor as disclosed above
c. and
d. applying the metallic precursor to the surface to deposit the conductive metallic film onto the substrate.

The deposition method is selected from the group consisting of spray coating, roll coating, doctor blade drawdown (squeegee), spin coating, pooling on the surface, condensation of supersaturated vapors, inkjet printing, curtain coating, dip-coating, and the combinations thereof.

When the metallic precursor is a liquid, it is applied to the surface with a contact angle between the metallic precursor and the surface at ≤90°, preferably ≤45°, or more preferably ≤30°.

The method can further comprises applying an energy to the metallic precursor to dissociate the ligands stabilizing the metal; and the energy is selected from the group consisting of visible, infrared or ultraviolet light; a heated gas stream; conduction from a resistively or fluid-heated susceptor; an induction-heated susceptor; electron beams; ion beams; remote hydrogen plasma; direct argon; helium or hydrogen plasma; vacuum; ultrasound; and combinations thereof.

The method can additionally comprises applying a post-deposition annealing treatment.

In another aspect, described herein is a system to deposit a conductive metallic film onto a substrate comprising:
a. the substrate with a surface containing topography;
b. the metallic precursor as disclosed above; and
c. a deposition tool selected from the group consisting of spray coating, roll coating, doctor blade drawdown (squeegee), spin coating, pooling on the surface, condensation of supersaturated vapors, inkjet printing, curtain coating, dip-coating, and the combinations thereof.

In yet another aspect, described herein is a vessel containing the metallic precursor as disclosed above. The vessel can have a dip-tube extending beneath the surface of the liquid metallic precursor to facilitate the delivering of the precursor to the deposition site.

In yet another aspect, described herein is a conductive metallic film deposited on a surface containing topography by using liquid metallic precursor and method disclosed above. The conductive metallic film has an electrical conductivity less or equal 1×10⁻⁴ Ωcm at ambient temperature.

### BRIEF DESCRIPTION OF THE DRAWINGS

The present invention will hereinafter be described in conjunction with the appended figures wherein like numerals denote like elements:

FIG. 1 shows thermogravimetric analysis (TGA) data for (1-decyne)tetracobalt dodecacarbonyl measured under flowing nitrogen.

FIG. 2 shows a typical conductive cobalt-containing film deposited on a wafer coupon in current application.

### DETAILED DESCRIPTION

The ensuing detailed description provides preferred exemplary embodiments only, and is not intended to limit the scope, applicability, or configuration of the invention. Rather, the ensuing detailed description of the preferred exemplary embodiments will provide those skilled in the art with an enabling description for implementing the preferred exemplary embodiments of the invention. Various changes may be made in the function and arrangement of elements without departing from the spirit and scope of the invention, as set forth in the appended claims.

In the claims, letters may be used to identify claimed method steps (e.g. a, b, and c). These letters are used to aid in referring to the method steps and are not intended to indicate the order in which claimed steps are performed, unless and only to the extent that such order is specifically recited in the claims.

The present invention uses a neutral (uncharged) metal compound as the precursor in which the metal atom is in the zerovalent state and stabilized by ligands which are stable as uncharged, volatile species in order to deposit a conductive metallic film on a surface which contains topography.

In order to create conductive paths on a surface which has been patterned with recesses in a dielectric material; a liquid metallic precursor containing a metallic compound as a liquid or as a solution in a suitable solvent is applied to the surface. The pool of liquid may be spread on the surface under inert conditions in a known manner so that the recessed areas are filled with this liquid by capillary action, optionally with excess liquid retained on top of the surface by the surface tension of the liquid. The substrate is then subjected to heating that leads to evaporation of the optional solvent and some of the stabilizing ligands, leading to partial decomposition of the precursor to form agglomerated metallic clusters or nanoparticles that on further heating coalesce in the recesses while they release the bulk of the stabilizing ligands to leave a conductive metallic solid.

This method is particularly advantageous when said topography or feature has a high aspect ratio. The aspect ratio (the depth to width ratio) of the surface features, if present, is 4:1 or greater, or 8:1 or greater, or 10:1 or greater, or 20:1 or greater, or 40:1 or greater.

The neutral (uncharged) metal compound can most advantageously be a liquid or a solid which melts at a temperature below its decomposition temperature or which has high solubility in a suitable solvent.

The metallic precursor comprises the neutral (uncharged) metal compound or the neutral (uncharged) metal compound with the solvent.

In order to facilitate transport of the metallic precursor into the topography on the surface, it is should be in the form of a low viscosity liquid.

If the neutral (uncharged) metal compound is a solid or viscous liquid at ambient temperature, it may conveniently be supplied as a solution in a suitable solvent. The viscosity of this liquid at ambient temperature should be between 0.5 cP and 20 cP, preferably between 1 cP and 10 cP and most preferably between 2 cP and 5 cP.

Suitable metals for the neutral (uncharged) metal precursor include all elements of the transition metal series, especially Fe, Co, Ni, Ru, Ir, Rh, Pd, Pt, Cu, Ag, Au, Os and combinations thereof.

Suitable ligands include, but are not limited to: carbon monoxide (CO), nitric oxide (NO), dinitrogen (N₂), acetylene (C₂H₂), ethylene (C₂H₄), dienes, trienes, tetraenes, cyclic dienes, organoisocyanides RNC wherein R═C₁ to C₁₂ linear branched hydrocarbyl or halocarbyl radical; organic nitriles RCN wherein R═C₁ to C₁₂ hydrocarbyl or halocarbyl radical; organophosphines PR′₃ wherein R′═H, Cl, F, Br, or a C₁ to C₁₂ hydrocarbyl or halocarbyl radical; amines NRaRbRc wherein Ra, Rb and Rc can be independently selected from H or a C₁ to C₁₂ hydrocarbyl or halocarbyl radical where they may be connected to each other; organic ethers with general formula R*OR** wherein R* and R** can be selected independently from C₁ to C₁₂ hydrocarbyl or halocarbyl radicals and may be connected to each other; and terminal or internal alkynes with general formula R₁CCR₂ where R₁ and R₂ can be independently selected from H, C₁ to C₁₂ linear, branched, cyclic or aromatic halocarbyl or hydrocarbyl radical, silyl or organosilyl radical (e.g. Si(CH₃)₃), SiCl₃), stannyl or organostannyl radical.

Examples of terminal or internal alkynes include but are not limited to propyne, 1-butyne, 3-methyl-1-butyne, 3,3-dimethyl-1-butyne, 1-pentyne, 1-hexyne, 1-decyne, cyclohexylacetylene, phenylacetylene, 2-butyne, 3-hexyne, 4,4-dimethyl-2-pentyne, 5,5-dimethyl-3-hexyne, 2,2,5,5-tetramethyl-3-hexyne, trimethysilylacetylene, phenyacetylene, diphenyl acetylene, trichlorosilylacetylene, trifluoromethylacetylene, cyclohexylacetylene, trimethylstannylacetylene.

Examples of organophosphines include but are not limited to phosphine (PH₃), phosphorus trichloride (PCl₃), phosphorus trifluoride (PF₃), trimethylphosphine (P(CH₃)₃), triethylphosphine (P(C₂H₅)₃), tributylphosphine (P(C₄H₉)₃), triphenylphosphine (P(C₆H₅)₃), tris(tolyl)phosphine (P(C₇H₇)₃), dimethylphosphinoethane ((CH₃)₂PCH₂CH₂P(CH₃)₂), diphenylphosphinoethane ((C₆H₅)₂PCH₂CH₂P(C₆H₅)₂).

Examples of organic isocyanides include but are not limited to methylisocyanide (CH₃NC), ethylisocyanide (C₂H₅NC), t-butylisocyanide ((CH₃)₃CNC), phenylisocyanide (C₆H₅NC), tolylisocyanide (C₇H₇NC), trifluoromethylisocyanide (F₃CNC).

Examples of amines include but are not limited to ammonia (NH₃), Trimethylamine ((CH₃)₃N), piperidine, ethylenediamine, pyridine.

Examples of ethers include but are not limited to dimethylether (CH₃OCH₃), diethylether (C₂H₅OC₂H₅), methyltertbutylether (CH₃OC(CH₃)₃), tetrahydrofuran, furan, ethyleneglycoldimethylether (CH₃OCH₂CH₂OCH₃), diethyleneglycoldimethylether (CH₃OCH₂CH₂OCH₂CH₂OCH₃).

Examples of organic nitriles include but are not limited to acetonitrile (CH₃CN), propionitrile (C₂H₅CN), benzonitrile (C₆H₅CN) and acrylonitrile (C₂H₃CN).

Examples of neutral (uncharged) metal precursors include but are not limited to R¹Co₂(CO)₆ wherein R¹ is a linear or branched C₂ to C₁₀ alkyne, a linear or branched C₁ to C₁₀ alkoxy alkyne, a linear or branched C₁ to C₁₀ organoamino alkyne such as (tert-butylacetylene)dicobalt hexacarbonyl [Co₂(CO)₆HC:::CC(CH₃)₃], R¹CoFe(CO)₇ wherein R¹ is a linear or branched C₂ to C₁₀ alkyne, a linear or branched C₁ to C₁₀ alkoxy alkyne, a linear or branched C₁ to C₁₀ organoamino alkyne, R²CCo₃(CO)₉ wherein R² is selected from the group consisting of hydrogen, a linear or branched C₁ to C₁₀ alkyl, a linear or branched C₁ to C₁₀ alkoxy, Cl, Br, COOH, COOMe, COOEt, R²CCo₂Mn(CO)₁₀ wherein R² is selected from the group consisting of hydrogen, a linear or branched C₁ to C₁₀ alkyl, a linear or branched C₁ to C₁₀ alkoxy, Cl, Br, COOH, COOMe, COOEt, R³Co₄(CO)₁₂ wherein R³ is selected from a linear or branched C₁ to C₁₀ alkenylidene, R⁴Ru₃(CO)₁₁ wherein R⁴ is selected from a disubstituted alkyne (R^{#}CCR^{##}) wherein R^{#} and R^{##} can be selected independently from C₁ to C₁₂ linear, branched, cyclic or aromatic halocarbyl or hydrocarbyl radical, silyl or organosilyl radical (e.g. Si(CH₃)₃), SiCl₃), stannyl or organostannyl radical, and combinations thereof.

Examples of neutral (uncharged) metal precursors include more specifically but are not limited todicobalthexacarbonyltert-butylacetylene [Co2(CO)6HC:::CC(CH3)3], (1-decyne) tetracobalt dodecacarbonyl (Co₄(CO)₁₂(C₈H₁₇C:::CH)), (1,6-Heptadiyne) tetracobalt dodecacarbonyl, (2,2,6-Trimethyl-3-heptyne) dicobalt hexacarbonyl, (2,2-Dimethyl-3-octyne) dicobalt hexacarbonyl (CCTNBA), (2,2-Dimethyl-3-decyne) dicobalt hexacarbonyl, (2,2-Dimethyl-3-heptyne) dicobalt hexacarbonyl, (tert-butylmethylacetylene)dicobalt hexacarbonyl (CCTMA), trirutheniumdodecacarbonyl, (ethylbenzene)(1,3-butadiene)Ruthenium, (isopropyl-4-methyl-Benzene)(1,3-butadiene)ruthenium, 1,3,5-cycloheptatrienedicarbonylruthenium, 1,3-cyclohexadienetricarbonylruthenium, 2,3-dimethyl-1,3-butadienetricarbonylruthenium, 2,4-hexadienetricarbonylruthenium, 1,3-pentadienetricarbonylruthenium, (benzene)(1,3-butadiene)ruthenium, (benzene)(2,3-Dimethyl-1,3-butadiene)ruthenium, Co₂Ru(CO)₁₁, HCoRu₃(CO)₁₃, Ru₃(CO)₉(PPh₂(CH₂)₃Si(OEt)₃)₃, bis(benzene)chromium, bis(cyclooctadiene)nickel, bis(tri-tert-butylphosphine)platinum, and bis(tri-tert-butylphosphine)palladium.

Some of the precursor as described above may be dissolved in a suitable solvent to render it into a low viscosity liquid.

Suitable solvents include but are not limited to saturated linear, branched and cyclic hydrocarbons.

Suitable solvents include but are not limited to n-hexane, n-pentane, isomeric hexanes, octane, isooctane, decane, dodecane, heptane, cyclohexane, methylcyclohexane, ethylcyclohexane, decalin; aromatic solvents such as benzene, toluene, xylene (single isomer or mixture of isomers), mesitylene, o-dichlorobenzene, nitrobenzene; nitriles such as acetonitrile, propionitrile or benzonitrile; ethers such as tetrahydrofuran, dimethoxyethane, diglyme, tetrahydropyran, methyltetrahydrofuran, butyltetrahydrofuran, p-dioxane; amines such as triethylamine, piperidine, pyridine, pyrrolidine, morpholine; amides such as N,N-dimethylacetamide, N,N-dimethylformamide, N-methylpyrrolidinone, N-cyclohexylpyrrolidinone; aminoethers having formaulae R⁴R⁵NR⁶OR⁷NR⁸R⁹, R⁴OR⁶NR⁸R⁹, O(CH₂CH₂)₂NR⁴, R⁴R⁵NR⁶N(CH₂CH₂)₂O, R⁴R⁵NR⁶OR⁷N(CH₂CH₂)₂O, O(CH₂CH₂)₂NR⁴OR⁶N(CH₂CH₂)₂O wherein R⁴⁻⁹ are independently selected from the group consisting of a linear or branched C₁ to C₁₀ alkyl and mixtures thereof.

The neat precursor liquid or a solution of precursor in solvent may be applied to a substrate having topographic features by means known in the art, including spray coating, roll coating, doctor blade drawdown (squeegee), spin coating, pooling on the surface, condensation of supersaturated vapors, inkjet printing, curtain coating, dip-coating or the like.

In order to achieve high quality films, the liquid may be applied to the substrate under a controlled atmosphere which has reduced oxygen or moisture content compared to ambient air. To enable such a process, the metal element containing liquids of the present invention can be contained in a sealed vessel or container, such as the one disclosed in US2002108670A1, the contents of which are incorporated herein by reference.

The vessel may be connected to deposition equipment known in the art by use of a valved closure and a sealable outlet connection. For convenience, the outlet connection may be connected to a dip-tube extending beneath the surface of the liquid so that the liquid may be delivered to the substrate by the use of a pressure difference.

Most preferably, the vessels may be constructed of high purity materials, including stainless steel, glass, fused quartz, polytetraflurorethylene, PFA®, FEP®, Tefzel® and the like. The vessels may be sealed with one or more valves. The headspace of the vessel is preferably filled with a suitable gas such as nitrogen, argon, helium or carbon monoxide. One or more of the valves may be connected to a dip tube which extends below the surface of the liquid, and one or more of the valves may be in fluid communication with the head space gas.

The liquid applied to the surface will be drawn into the fine topography on the surface due to capillary action. In order to fill fine topographic features, therefore, a contact angle between this liquid and the surface(s) being coated needs to be ≤90°, preferably ≤45°, or more preferably ≤30°.

Contact angle is one of the common ways to measure the wettability of a surface or material. Wetting refers to the study of how a liquid deposited on a substrate spreads out or the ability of liquids to form boundary surfaces with the substrate. The wetting is determined by measuring the contact angle, which the liquid forms in contact with the substrate. The wetting tendency is larger, the smaller the contact angle or the surface tension is. A wetting liquid is a liquid that forms a contact angle with the solid which is smaller than 90°, whereas, a nonwetting liquid creates a contact angle between 90 and 180 with the solid.

In order for such filling to take place at a reasonable rate, the viscosity of the liquid at ambient temperature should be between 0.5 cP and 20 cP, preferably between 1 cP and 10 cP and most preferably between 2 cP and 5 cP.

In the next step, energy is applied to the liquid precursor, causing dissociation of the neutral ligands stabilizing the metal. As these ligands dissociate, the metal ions will begin to coalesce, forming small agglomerates or clusters. As the optional solvent evaporates and more ligands dissociate, these agglomerates continue to grow and concentrate. As these metallic clusters grow, they become nanometer scale particles (nanoparticles). The nanoparticles will concentrate in the recesses of the topography as the solvent and unreacted zerovalent metal-organic liquid evaporate. Then, a conductive film is formed.

A conductive film should have an electrical conductivity at ambient temperature less than or equal (≤) about 1×10⁻⁴ Ωcm. For a 100 Å thick film, this corresponds to a measured sheet resistance less than about 100 Ω/square.

Resistivity of the conductive deposit may be improved by applying energy to the deposited material. Energy is most conveniently applied by external heating using visible or infrared or ultraviolet light or a combination of these radiation sources, through convection using a heated gas stream or by conduction from a resistively or fluid-heated susceptor or from an induction-heated susceptor on which the substrate is placed.

Other sources of energy might also be useful for this process, including electron beams, ion beams, remote hydrogen plasma, direct argon, helium or hydrogen plasma, vacuum and ultrasound.

The conductive film can be further undergo a post-deposition annealing treatment.

The post-deposition annealing treatment can be carried out under a reducing atmosphere, including but not limited to hydrogen, ammonia, diborane, silane, at a temperature at or above (≥) 300° C., for example, from 300° C. to 700° C.; with annealing time of or more than (≥) 5 minutes, for example from 5 to 60 minutes.

The reducing atmospheres can be pure reducing gases or mixtures of the reducing gases with inert gases such as nitrogen or argon. The pressure of the reducing atmosphere can be at or above (≥) 10 torr, for example, range from 10 torr to 760 torr; and the flow rate of the reducing gas can be at or above (≥) 100 sccm, for example, range from 100-1000 sccm.

In another aspect, the present invention is also a vessel or container employing the metallic precursor comprises at least one neutral (uncharged) metal precursor or at least one neutral (uncharged) metal precursor with a solvent.

The method described herein may be used to deposit a conductive film on at least a portion of a substrate. Examples of suitable semiconductor substrates include but are not limited to, silicon, SiO₂, Si₃N₄, OSG, FSG, silicon carbide, hydrogenated silicon oxycarbide, hydrogenated silicon oxynitride, silicon carbo-oxynitride, hydrogenated silicon carbo-oxynitride, antireflective coatings, photoresists, germanium, germanium-containing, boron-containing, Ga/As, a flexible substrate, organic polymers, porous organic and inorganic materials, metals such as copper and aluminum, metal silicide such as titanium silicide, tungsten silicide, molybdenum silicide, nickel silicide, cobalt silicide, and diffusion barrier layers such as but not limited to cobalt, TiN, Ti(C)N, TaN, Ta(C)N, Ta, W, or WN.

### EXAMPLES

### Example 1

A silicon wafer has a surface layer of carbon-doped silicon oxide into which trenches that are 20 nm wide and 200 nm deep have been etched.

The silicon wafer is situated on a platform in a sealed chamber under inert conditions in a dry oxygen-free nitrogen environment.

Liquid dicobalthexacarbonyltert-butylacetylene (Co₂(CO)₆HC:::CC(CH₃)₃) as the precursor is placed on the silicon wafer.

The pressure of the chamber is reduced first so that any N₂ trapped in the trenches can be removed and the liquid can flow into the trenches by capillary action.

The pressure is then increased by adding nitrogen and then the temperature of the platform is increased gradually.

As the liquid begins to decompose t-butyl acetylene vapors and CO gas will be released and the precursor molecules will begin to oligomerize. The volume of the liquid contracts and the liquid residing on top of the trenches is drawn into the trenches. As condensation continues, solid nanoparticles might form and pack tightly in the trenches.

As the temperature reaches 400° C., most of the CO and tert-butylacetylene ligands will released into the vapor phase, leaving a conductive Co metal deposit mostly inside the trenches.

Further optional annealing of the deposited material with H₂ gas or by using plasma or electron beams can be employed at this point to increase the conductivity of the metal.

Conventional processing to remove overburden (excess Co on the upper surfaces) such as by chemical mechanical planarization (CMP) can then be performed.

If the trenches are not completely filled, the deposition process may be repeated one or more times until the trenches are completely filled with conductive cobalt metal.

### Example 2

A silicon wafer has a surface layer of carbon-doped silicon oxide into which trenches that are 20 nm wide and 200 nm deep have been etched.

The silicon wafer is situated on a platform in a sealed chamber under inert conditions in a dry oxygen-free nitrogen environment.

Liquid dicobalthexacarbonyltert-butylacetylene (Co₂(CO)₆HC:::CC(CH₃)₃) as the precursor combined with about 10 weight percent dry n-octane is placed on the silicon wafer.

The pressure of the chamber is reduced first so that any N₂ trapped in the trenches can be removed and the liquid can flow into the trenches by capillary action.

The pressure is then increased by adding nitrogen and then the temperature of the platform is increased gradually.

As the liquid begins to decompose t-butyl acetylene vapors and CO gas will be released and the precursor molecules will begin to oligomerize. The volume of the liquid contracts and the liquid residing on top of the trenches is drawn into the trenches. As condensation continues, solid nanoparticles might form and pack tightly in the trenches.

As the temperature reaches 400° C., most of the CO and tert-butylacetylene ligands will released into the vapor phase, leaving a conductive Co metal deposit mostly inside the trenches.

Further optional annealing of the deposited material with H₂ gas or by using plasma or electron beams can be employed at this point to increase the conductivity of the metal.

Conventional processing to remove overburden (excess Co on the upper surfaces) such as by chemical mechanical planarization (CMP) can then be performed.

If the trenches are not completely filled, the deposition process may be repeated one or more times until the trenches are completely filled with conductive cobalt metal.

### Example 3

### Synthesis of (1-decyne)tetracobalt Dodecacarbonyl (Co₄(CO)₁₂(C₈H₁₇C:::CH))

In a nitrogen glovebox, tetracobalt dodecacarbonyl (500 mg, 0.87 mmol) was placed in a 25 cc Schlenk flask. 10 mL Tetrahydrofuran was added into the flask.

Upon stirring, the tetracobalt dodecacarbonyl dissolved to yield a dark solution. 1-Decyne (550 mg, 4.0 mmol) was added to the solution.

The solution was stirred at ambient temperature for 2 days. During this time, the color of the solution changed to dark red.

The volatiles were removed under vacuum to yield a highly viscous black liquid.

### Example 4

### Thermal Decomposition of (1-decyne)tetracobalt Dodecacarbonyl

In a nitrogen glovebox, a sample of (1-decyne)tetracobalt dodecacarbonyl was placed on a flat pan and transferred to a Thermogravimetric analyzer(TGA).

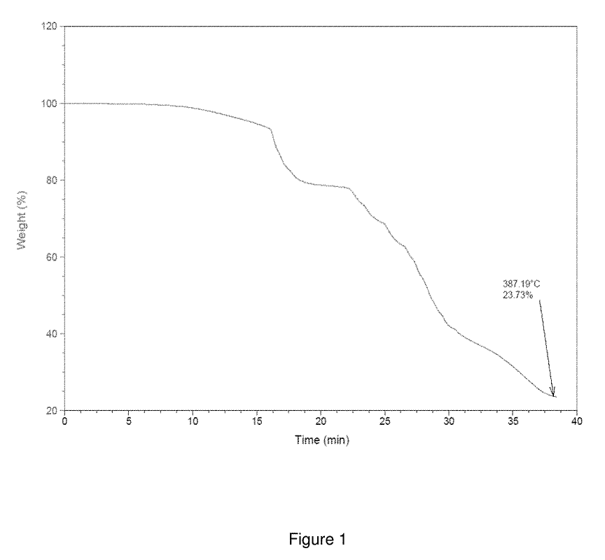

Using the TGA, the temperature of the sample was ramped to 400° C. at 10° C./minute while monitoring the weight of the sample. A total of 76% of the initial weight was lost, leaving 24% residue (FIG. 1). In the compound (1-decyne)tetracobalt dodecacarbonyl, cobalt makes up about 33% of the mass and the ligands make up about 67%. Thus, a majority of the cobalt initially present in the mixture is retained on the surface of the pan.

### Example 5

### Synthesis of Ru₃(CO)₉(PPh₂(CH₂)₃Si(OEt)₃)₃ as a Precursor

Ru₃(CO)₁₂ (0.5 g, 0.78 mmol) from Colonial metals inc. and PPh₂(CH₂)₃Si(OEt)₃ (1 g, 2.56 mmol) from Strem Chemicals are charged into a 250 ml flask inside the glovebox. The flask is then moved out of the glovebox and attached to Schlenk line (under N₂).

Under N₂ purge and stirring, anhydrous hexane (100 mL) from Sigma-Aldrich is added into the flask with a syringe. The flask is heated under reflux for two hours at 68-70° C. After two hours, the reaction is cooled down to ambient temperature. All solvent is pumped off under vacuum at ambient temperature. The product is washed by cold hexane 3×10 ml. The final product is dried under vacuum. Reddish oil, 0.55 g, yield 85% is then obtained.

### Example 6

A mixture of triruthenium dodecacarbonyl with 20% dry n-octane is placed on a silicon wafer having a surface layer of carbon-doped silicon oxide into which trenches that are 20 nm wide and 200 nm deep have been etched. The wafer is sealed in a chamber under inert conditions in a dry oxygen-free nitrogen environment. The pressure of the chamber is reduced so that any N₂ trapped in the trenches can be removed and the liquid can flow into the trenches by capillary action while the solvent begins to evaporate. The pressure is then increased by adding nitrogen and then the temperature of the platform on which the wafer is situated is increased gradually. As the liquid begins to decompose, decyne vapors and CO gas will be released and the precursor molecules will begin to oligomerize. The volume of the liquid contracts and the liquid residing on top of the trenches is drawn into the trenches. As condensation continues, solid nanoparticles might form and pack tightly in the trenches. As the temperature reaches 400° C., most of the CO ligands will released into the vapor phase, leaving a conductive ruthenium metal deposit mostly inside the trenches. Further optional thermal annealing of the deposited material with H₂ or C₂ gas or by using plasma or electron beams can be employed at this point to increase the conductivity of the metal. Conventional processing to remove overburden (excess Ru on the upper surfaces) such as by chemical mechanical planarization (CMP) can then be performed. If the trenches are not completely filled, this process may be repeated one or more times until the trenches are completely filled with conductive ruthenium or a different metal.

### Example 7

(1,6-Heptadiyne) tetracobalt dodecacarbonyl combined with about 10 weight percent dry n-octane is placed on a silicon wafer having a surface layer of carbon-doped silicon oxide into which trenches that are 20 nm wide and 200 nm deep have been etched. The wafer is sealed in a chamber under inert conditions in a dry oxygen-free nitrogen environment. The pressure of the chamber is reduced so that any N₂ trapped in the trenches can be removed and the liquid can flow into the trenches by capillary action while the solvent begins to evaporate. The pressure is then increased by adding nitrogen and then the temperature of the platform on which the wafer is situated is increased gradually. As the liquid begins to decompose, 1,6-Heptadiyne vapors and CO gas will be released and the precursor molecules will begin to oligomerize. The volume of the liquid contracts and the liquid residing on top of the trenches is drawn into the trenches. As condensation continues, solid nanoparticles might form and pack tightly in the trenches. As the temperature reaches 400° C., most of the CO and 1,6-Heptadiyne ligands will released into the vapor phase, leaving a conductive Co metal deposit mostly inside the trenches. Further optional annealing of the deposited material with H2 gas or by using plasma or electron beams can be employed at this point to increase the conductivity of the metal. Conventional processing to remove overburden (excess Co on the upper surfaces) such as by chemical mechanical planarization (CMP) can then be performed. If the trenches are not completely filled, this process may be repeated one or more times until the trenches are completely filled with conductive cobalt metal.

### Example 8

### Synthesis of 2,2-Dimethyl-3-octyne (tert-butyl n-butyl acetylene)

In a nitrogen glovebox, a solution of tert-butylacetylene (3,3-Dimethyl-1-butyne) was prepared by placing tert-butylacetylene (32.8 g, 0.4 mol) in a 1000 mL round bottom flask with 500 mL of anhydrous THF. To a 500 mL addition funnel was added 150 mL of 2.5 M n-Butyllithium in hexanes (0.375 mol). The flask and addition funnel were removed from the glovebox and assembled in the hood. The tert-butylacetylene solution was cooled to 0° C. The n-Butyllithium solution was added dropwise to the tert-butylacetylene solution over 30 minutes with stirring. After the addition was complete, the colorless solution was allowed to warm to ambient temperature over two hours with stirring. To a 500 mL addition funnel was added 1-lodobutane (64.4 g, 0.35 mol) and 100 mL anhydrous THF. This solution was added dropwise to the lithium tert-butylacetylide solution over 30 minutes with stirring. The solution was stirred at ambient temperature for 3 days. GC-MS analysis of a small sample showed complete conversion to the product. The solution was extracted two times with 100 mL of deionized water. The water washes were extracted with 200 mL of hexane and this extract was combined with the THF/hexane solution. The organic solution was dried over magnesium sulfate for 30 minutes. During this time, the colorless solution became light yellow. The combined organic solutions were distilled at reduced pressure (˜10 Torr) while holding the reboiler at 20° C., the condenser at 0° C., and the collection flask at −78° C. After the removal of solvent, another collection flask was fitted, and the remaining volatiles distilled while holding the reboiler at 25° C., the condenser at 0° C., and the collection flask at −78° C. The pressure during the second distillation was ˜2 torr. When all of the volatiles had been transferred, the collection flask was allowed to warm to ambient temperature. The colorless liquid was analyzed using GC-MS, confirming the presence of highly pure product (≥99% purity, 42.2 g, 87% yield).

¹H NMR analysis of 2,2-Dimethyl-3-octyne gives the following chemical shifts: 2.03 (t, 2H); 1.33 (m, 4H); 1.19 (s, 9H); 0.80 (t, 3H).

### Example 9

### Synthesis of (2,2-Dimethyl-3-octyne) Dicobalt Hexacarbonyl (Cobalt Carbonyl Tert-butyl N-Butyl Acetylene, CCTNBA)

In a ventilated hood, a solution of 2,2-Dimethyl-3-octyne (21.5 g, 0.15 mol) in hexanes (100 mL) was added over 30 minutes to a solution of Co₂(CO)₈ (47.5 g, 0.14 mol) in hexanes (700 mL). Visible CO evolution was observed upon addition of the 2,2-Dimethyl-3-octyne solution. The resulting dark brown solution turned dark reddish brown over the course of stirring at ambient temperature for four hours. The hexanes were removed using vacuum distillation while holding the reboiler at 25° C. (condenser temp. −5° C.; collection flask temp. −78° C.), to yield a dark red liquid with dark solids. A chromatography column (˜3 inches in diameter) was packed with 8 inches of neutral activated alumina using pure hexanes as the eluent. The crude material was placed on the column and eluted using hexanes. A brown band quickly moved down the column with the hexanes. Dark purple material was retained in the top 2-3″ of the column. The reddish-brown band was collected and evacuated on a Schlenk line (˜700 mTorr), yielding 40.0 g of a dark red liquid.

¹H NMR analysis of CCTNBA showed high purity (NMR assay 99.6%). Chemical shifts (d₈-toluene): 2.66 (t, 2H), 1.60 (m, 2H), 1.29 (m, 2H), 1.17 (s, 9H), 0.86 (t, 3H).

### Example 10

### Formation of Cobalt-Containing Films Using CCTNBA

In a nitrogen glovebox, ˜20 wt. % solutions of CCTNBA were prepared in hexanes and toluene by weighing 250 mg of CCTNBA and 1 g of hexanes/toluene into two 25 mL glass bottles.

Wafer coupons of thermal SiO₂ and silicon of approximate dimensions of 1″×1″ were brought into a nitrogen glovebox. Two coupons of each type were placed in a glass evaporating dish.

The coupons were covered with a thin film of either solution with CCTNBA in hexanes or solution with CCTNBA in toluene by adding the solutions dropwise to the surfaces of the coupons.

The wetting properties of the solutions were slightly different. It took about 5-6 drops of the solution having hexanes s to cover the entire coupon surface. It took 8-9 drops of the solution having toluene to cover the entire coupon surface.

For both sets of solutions, it was possible to cover essentially the entire surface area of the coupons without any of the solutions spilling over the edges of the coupons.

The coupons with the ˜20 wt. % solutions of CCTNBA were allowed to stand at room temperature in the glovebox. During this time, the hexanes solutions evaporated entirely. However, the toluene solutions were only partially evaporated.

The glass dish containing the coupons was carefully placed on a heating plate. The heating plate was warmed to 80 deg. C. After several minutes, it was apparent that the toluene had evaporated and the CCTNBA was still present on the coupon surfaces. After 5 minutes, the dish was removed from the heating plate.

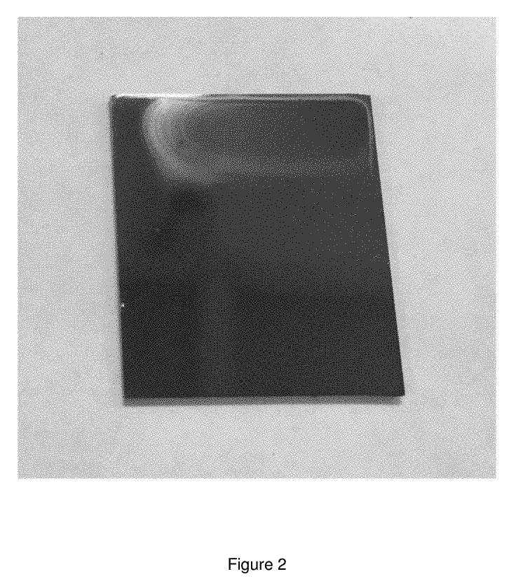

The temperature of the hotplate was increased to 370 deg. C. When the hotplate surface was stabilized at 370 deg. C., the dish containing the coupons was placed back on the hotplate. A second evaporating dish of a slightly larger size was placed on top of the dish containing the coupons (acting as a lid). After about 30 seconds, a small amount brown vapor was observed rising from the coupon surfaces. The vapor condensed on the sides of the dish containing the coupons and the part of the larger dish acting as a lid. The coupons were heated for 15 minutes at 370 deg. C. Within several minutes at 370 deg. C., the coupon surfaces were mostly shiny silver with some dull grey regions. The hotplate heating was terminated, the glass dish was allowed to cool to ambient temperature. The conductive cobalt-containing films were deposited on the coupons. An example was shown in FIG. 2.

The coupons were removed from the dish for analysis.

X-ray fluorescence (XRF) was used to measure the film thickness. A four-point probe was used to measure the film sheet resistance. The sheet resistance was measured after film deposition. The results were shown in Table 1.

The coupons were then placed in a chamber for annealing under a hydrogen-containing atmosphere. The conditions for post-deposition annealing treatment were: nitrogen flow 450 sccm, hydrogen flow 50 sccm, temperature 400° C., chamber pressure 50 torr, anneal time 30 minutes.

The four-point probe was used again to measure the film sheet resistance after the annealing. The results were shown in Table 1.

Table I shows the effect of annealing on the resistivity of the deposited cobalt films. The annealing process lowers the resistivity of the cobalt-containing films.

TABLE I

| | | | Sheet | Sheet |
|---|---|---|---|---|
| | | | resistance | resistance |
| | | Film | before H₂ | after H₂ |
| Wafer | | thickness | anneal | anneal |
| surface | Solvent | (Angstroms) | (ohms/sq) | (ohms/sq) |
| SiO₂ | Hexanes | 196 | 1300 | 1090 |
| SiO₂ | Toluene | 515 | 2420 | 2160 |
| SiO₂ | Hexanes | 236 | 6790 | 4700 |
| SiO₂ | Toluene | 260 | 1220 | 218 |
| Si | Hexanes | 690 | 154 | 9 |
| Si | Toluene | 618 | 476 | 189 |
| Si | Hexanes | 197 | Not | 197 |
| | | | conductive | |
| Si | Toluene | 668 | 494 | 33 |

Films were deposited on both silica and silicon surfaces. Most of the films as deposited contain cobalt and were conductive as measured by a four-point probe measurement apparatus. There appeared to be impurities, such as carbon, in the cobalt films that result in high sheet resistance. Annealing the cobalt films under a reducing atmosphere, such as a mixture of hydrogen and nitrogen, is a method of reducing impurity levels.

The results in Table I demonstrate that the resistivity can be lowered in the films of the current invention. The resulting films may be used to generate a conductive layer or conductive features, such as conductive lines or vias, in semiconductor devices.

While the principles of the invention have been described above in connection with preferred embodiments, it is to be clearly understood that this description is made only by way of example and not as a limitation of the scope of the invention.

1. A method to deposit a conductive metallic film onto a substrate comprising:
a. providing the substrate with a surface containing topography;
b. providing liquid metallic precursor comprising a neutral (uncharged) metal compound having a metal in zerovalent state and at least one neutral stabilizing ligand;
wherein
the metal is selected from the group consisting of Fe, Co, Ni, Ru, Ir, Rh, Pd, Pt, Cu, Ag, Au, Os, and combinations thereof;
the at least one neutral stabilizing ligand is selected from the group consisting of
carbon monoxide (CO); nitric oxide (NO); dinitrogen (N₂); acetylene (C₂H₂); ethylene (C₂H₄); C₄-C₁₈ diene or C₄-C₁₈ cyclic diene; C₆-C₁₈ triene; C₈-C₁₈ tetraene; organo isocyanide RNC, wherein R is selected from the group consisting of C₁ to C₁₂ linear or branched hydrocarbyl or halocarbyl radical; organic nitrile RCN wherein R is selected from the group consisting of C₁ to C₁₂ hydrocarbyl or halocarbyl radical; organophosphine PR′3 wherein R′ is selected from the group consisting of H, Cl, F, Br, and a C₁ to C₁₂ hydrocarbyl or halocarbyl radical; amine NRaRbRc, wherein Ra, Rb and Rc may be connected to each other and each is independently selected from H or a C₁ to C₁₂ hydrocarbyl or halocarbyl radical; organic ether R*OR**, wherein R* and R** can be connected to each other and each is selected independently from C₁ to C₁₂ hydrocarbyl or halocarbyl radicals; and terminal or internal alkyne with general formula R₁CCR₂, where R₁ and R₂ can be independently selected from the group consisting of H, C₁ to C₁₂ linear, branched, cyclic or aromatic halocarbyl or hydrocarbyl radical, silyl or organosilyl radical, stannyl or organostannyl radical, and combinations thereof;
the neutral (uncharged) metal compound is a liquid or a solid soluble at ambient temperature in a solvent selected from the group consisting of saturated linear, branched and cyclic hydrocarbons; or is a solid which melts at a temperature below its decomposition temperature;
and
the liquid metallic precursor has a viscosity at ambient temperature between 0.5 cP and 20 cP;
and
c. applying the liquid metallic precursor to the surface to deposit the conductive metallic film onto the substrate by spray coating, roll coating, doctor blade drawdown (squeegee), spin coating, pooling on the surface, condensation of supersaturated vapors, inkjet printing, curtain coating, dip-coating, or the combinations thereof.

2. The method of claim 1, wherein the neutral (uncharged) metal compound is selected from the group consisting of
a. R¹Co₂(CO)₆, wherein R¹ is a linear or branched C₂ to C₁₀ alkyne, a linear or branched C₁ to C₁₀ alkoxy alkyne, a linear or branched C₁ to C₁₀ organoamino alkyne such as (tert-butylacetylene)dicobalt hexacarbonyl;
[Co₂(CO)₆HC:::CC(CH₃)₃];
b. R¹CoFe(CO)₇, wherein R¹ is a linear or branched C₂ to C₁₀ alkyne, a linear or branched C₁ to C₁₀ alkoxy alkyne, a linear or branched C₁ to C₁₀ organoamino alkyne;
c. R²CCo₃(CO)₉, wherein R² is selected from the group consisting of hydrogen, a linear or branched C₁ to C₁₀ alkyl, a linear or branched C₁ to C₁₀ alkoxy, Cl, Br, COOH, COOMe, COOEt;
d. R²CCo₂Mn(CO)₁₀, wherein R² is selected from the group consisting of hydrogen, a linear or branched C₁ to C₁₀ alkyl, a linear or branched C₁ to C₁₀ alkoxy, Cl, Br, COOH, COOMe, COOEt;
e. R³Co₄(CO)₁₂, wherein R³ is selected from a linear or branched C₁ to C₁₀ alkenylidene; and
f. R⁴Ru₃(CO)₁₁, wherein R⁴ is selected from the group consisting of a disubstituted alkyne (R^{#}CCR^{##}) wherein R^{#} and R^{##} can be selected independently from the group consisting of C₁ to C₁₂ linear, branched, cyclic or aromatic halocarbyl or hydrocarbyl radical, silyl or organosilyl radical, stannyl or organostannyl radical, and combinations thereof.

3. The method of claim 1, wherein the neutral (uncharged) metal compound is selected from the group consisting of dicobalthexacarbonyltert-butylacetylene [Co₂(CO)₆HC:::CC(CH₃)₃], (1-decyne) tetracobalt dodecacarbonyl (Co₄(CO)₁₂(C₈H₁₇C:::CH)), (1,6-Heptadiyne) tetracobalt dodecacarbonyl, (2,2,6-Trimethyl-3-heptyne) dicobalt hexacarbonyl, (2,2-Dimethyl-3-octyne) dicobalt hexacarbonyl, (2,2-Dimethyl-3-decyne) dicobalt hexacarbonyl(CCTNBA), (2,2-Dimethyl-3-heptyne) dicobalt hexacarbonyl, (tert-butylmethylacetylene)dicobalt hexacarbonyl (CCTMA), trirutheniumdodecacarbonyl, (ethylbenzene)(1,3-butadiene)Ruthenium, (isopropyl-4-methyl-Benzene)(1,3-butadiene)ruthenium, 1,3,5-cycloheptatrienedicarbonylruthenium, 1,3-cyclohexadienetricarbonylruthenium, 2,3-dimethyl-1,3-butadienetricarbonylruthenium, 2,4-hexadienetricarbonylruthenium, 1,3-pentadienetricarbonylruthenium, (benzene)(1,3-butadiene)ruthenium, (benzene)(2,3-Dimethyl-1,3-butadiene)ruthenium, Co₂Ru(CO)₁₁, HCoRu₃(CO)₁₃, Ru₃(CO)₉(PPh₂(CH₂)₃Si(OEt)₃)₃, bis(benzene)chromium, bis(cyclooctadiene)nickel, bis(tri-tert-butylphosphine)platinum, bis(tri-tert-butylphosphine)palladium, and combinations thereof.

4. The method of claim 1, wherein the solvent is selected from the group consisting of n-hexane, n-pentane, isomeric hexanes, octane, isooctane, decane, dodecane, heptane, cyclohexane, methylcyclohexane, ethylcyclohexane, decalin; aromatic solvent selected from a group comprising of benzene, toluene, xylene (single isomer or mixture of isomers), mesitylene, o-dichlorobenzene, nitrobenzene; nitriles selected from a group comprising of acetonitrile, propionitrile or benzonitrile; ethers selected from a group comprising of tetrahydrofuran, dimethoxyethane, diglyme, tetrahydropyran, methyltetrahydrofuran, butyltetrahydrofuran, p-dioxane; amines selected from a group comprising of triethylamine, piperidine, pyridine, pyrrolidine, morpholine; amides selected from a group comprising of N,N-dimethylacetamide, N,N-dimethylformamide, N-methylpyrrolidinone, N-cyclohexylpyrrolidinone; aminoethers having formaulae R⁴R⁵NR⁶OR⁷NR⁸R⁹, R⁴OR⁶NR⁸R⁹, O(CH₂CH₂)₂NR⁴, R⁴R⁵NR⁶N(CH₂CH₂)₂O, R⁴R⁵NR⁶OR⁷N(CH₂CH₂)₂O, O(CH₂CH₂)₂NR⁴OR⁶N(CH₂CH₂)₂O; wherein R⁴⁻⁹ are independently selected from the group consisting of a linear or branched C1 to C₁₀ alkyl; and combinations thereof.

5. The method of claim 1, wherein
the neutral (uncharged) metal compound is selected from the group consisting of dicobalthexacarbonyltert-butylacetylene [Co₂(CO)₆HC:::CC(CH₃)₃], (1-decyne) tetracobalt dodecacarbonyl (Co₄(CO)₁₂(C₈H₁₇C:::CH)), (1,6-Heptadiyne) tetracobalt dodecacarbonyl, (2,2,6-Trimethyl-3-heptyne) dicobalt hexacarbonyl,
(2,2-Dimethyl-3-octyne) dicobalt hexacarbonyl(CCTNBA), and Ru₃(CO)₉(PPh₂(CH₂)₃Si(OEt)₃)₃;
and
the solvent is selected from the group consisting of tetrahydrofuran, octane, hexane, toluene.

6. The method of claim 1, wherein the liquid metallic precursor is applied to the surface with a contact angle between the liquid metallic precursor and the surface at ≤90°.

7. The method of claim 1, wherein the liquid metallic precursor has a viscosity at ambient temperature between 1 cP and 10 cP; and is applied to the surface with a contact angle between the liquid metallic precursor and the surface at <45°.

8. The method of claim 1 further comprises applying an energy to the liquid metallic precursor to dissociate the ligands stabilizing the metal; wherein the energy is selected from the group consisting of visible, infrared or ultraviolet light; a heated gas stream; conduction from a resistively or fluid-heated susceptor; an induction-heated susceptor; electron beams; ion beams; remote hydrogen plasma; direct argon; helium or hydrogen plasma; vacuum; ultrasound; and combinations thereof.

9. The method of claim 1 further comprises applying a post-deposition annealing treatment under a reducing atmosphere using a reducing gas selected from the group consisting of hydrogen, ammonia, diborane, silane, and combinations thereof for an annealing time of or more than 5 minutes;
wherein the reducing atmosphere is optionally further comprises an inert gas of nitrogen, argon or combinations of nitrogen and argon and the reducing atmosphere is at a temperature equal or above 300° C.; and the reducing gas is flowing at or above (≥) 100 sccm.

10. A system to deposit a conductive metallic film onto a substrate comprising:
a. the substrate with a surface containing topography;
b. liquid metallic precursor comprising a neutral (uncharged) metal compound having a metal in zerovalent state and at least one neutral stabilizing ligand;
wherein
the metal is selected from the group consisting of Fe, Co, Ni, Ru, Ir, Rh, Pd, Pt, Cu, Ag, Au, Os, and combinations thereof;
the at least one neutral stabilizing ligand is selected from the group consisting of
carbon monoxide (CO); nitric oxide (NO); dinitrogen (N₂); acetylene (C₂H₂); ethylene (C₂H₄); C₄-C₁₈ diene or C₄-C₁₈ cyclic diene; C₆-C₁₈ triene; C₈-C₁₈ tetraene; organo isocyanide RNC, wherein R is selected from the group consisting of C₁ to C₁₂ linear or branched hydrocarbyl or halocarbyl radical; organic nitrile RCN wherein R is selected from the group consisting of C, to C₁₂ hydrocarbyl or halocarbyl radical; organophosphine PR′3 wherein R′ is selected from the group consisting of H, Cl, F, Br, and a C₁ to C₁₂ hydrocarbyl or halocarbyl radical; amine NRaRbRc, wherein Ra, Rb and Rc may be connected to each other and each is independently selected from H or a C, to C₁₂ hydrocarbyl or halocarbyl radical; organic ether R*OR**, wherein R* and R** can be connected to each other and each is selected independently from C₁ to C₁₂ hydrocarbyl or halocarbyl radicals; and terminal or internal alkyne with general formula R₁CCR₂, where R₁ and R₂ can be independently selected from the group consisting of H, C₁ to C₁₂ linear, branched, cyclic or aromatic halocarbyl or hydrocarbyl radical, silyl or organosilyl radical, stannyl or organostannyl radical, and combinations thereof;
the neutral (uncharged) metal compound is a liquid or a solid soluble at ambient temperature in a solvent selected from the group consisting of saturated linear, branched and cyclic hydrocarbons; or is a solid which melts at a temperature below a decomposition temperature;
and
the liquid metallic precursor has a viscosity at ambient temperature between 0.5 cP and 20 cP;
and
c. a deposition tool selected from the group consisting of spray coating, roll coating, doctor blade drawdown (squeegee), spin coating, pooling on the surface, condensation of supersaturated vapors, inkjet printing, curtain coating, dip-coating, and the combinations thereof.

11. The system of claim 10, wherein the neutral (uncharged) metal compound is selected from the group consisting of
a. R¹Co₂(CO)₆, wherein R¹ is a linear or branched C₂ to C₁₀ alkyne, a linear or branched C₁ to C₁₀ alkoxy alkyne, a linear or branched C₁ to C₁₀ organoamino alkyne such as (tert-butylacetylene)dicobalt hexacarbonyl;
[Co₂(CO)₆HC:::CC(CH₃)₃];
b. R¹CoFe(CO)₇, wherein R¹ is a linear or branched C₂ to C₁₀ alkyne, a linear or branched C₁ to C₁₀ alkoxy alkyne, a linear or branched C₁ to C₁₀ organoamino alkyne;
c. R²CCo₃(CO)₉, wherein R² is selected from the group consisting of hydrogen, a linear or branched C₁ to C₁₀ alkyl, a linear or branched C₁ to C₁₀ alkoxy, Cl, Br, COOH, COOMe, COOEt;
d. R²CCo₂Mn(CO)₁₀, wherein R² is selected from the group consisting of hydrogen, a linear or branched C₁ to C₁₀ alkyl, a linear or branched C, to C₁₀ alkoxy, Cl, Br, COOH, COOMe, COOEt;
e. R³Co₄(CO)₁₂, wherein R³ is selected from a linear or branched C, to C₁₀ alkenylidene; and
f. R⁴Ru₃(CO)₁₁, wherein R⁴ is selected from the group consisting of a disubstituted alkyne (R^{#}CCR^{##}) wherein R^{#} and R^{##} can be selected independently from the group consisting of C₁ to C₁₂ linear, branched, cyclic or aromatic halocarbyl or hydrocarbyl radical, silyl or organosilyl radical, stannyl or organostannyl radical, and combinations thereof.

12. The system of claim 10, wherein the neutral (uncharged) metal compound is selected from the group consisting of dicobalthexacarbonyltert-butylacetylene [Co₂(CO)₆HC:::CC(CH₃)₃], (1-decyne) tetracobalt dodecacarbonyl (Co₄(CO)₁₂(C₈H₁₇C:::CH)), (1,6-Heptadiyne) tetracobalt dodecacarbonyl, (2,2,6-Trimethyl-3-heptyne) dicobalt hexacarbonyl, (2,2-Dimethyl-3-octyne) dicobalt hexacarbonyl, (2,2-Dimethyl-3-decyne) dicobalt hexacarbonyl(CCTNBA), (2,2-Dimethyl-3-heptyne) dicobalt hexacarbonyl, (tert-butylmethylacetylene)dicobalt hexacarbonyl (CCTMA), trirutheniumdodecacarbonyl, (ethylbenzene)(1,3-butadiene)Ruthenium, (isopropyl-4-methyl-Benzene)(1,3-butadiene)ruthenium, 1,3,5-cycloheptatrienedicarbonylruthenium, 1,3-cyclohexadienetricarbonylruthenium, 2,3-dimethyl-1,3-butadienetricarbonylruthenium, 2,4-hexadienetricarbonylruthenium, 1,3-pentadienetricarbonylruthenium, (benzene)(1,3-butadiene)ruthenium, (benzene)(2,3-Dimethyl-1,3-butadiene)ruthenium, Co₂Ru(CO)₁₁, HCoRu₃(CO)₁₃, Ru₃(CO)₉(PPh₂(CH₂)₃Si(OEt)₃)₃, bis(benzene)chromium, bis(cyclooctadiene)nickel, bis(tri-tert-butylphosphine)platinum, bis(tri-tert-butylphosphine)palladium, and combinations thereof.

13. The system of claim 10, wherein the solvent is selected from the group consisting of n-hexane, n-pentane, isomeric hexanes, octane, isooctane, decane, dodecane, heptane, cyclohexane, methylcyclohexane, ethylcyclohexane, decalin; aromatic solvent selected from a group comprising of benzene, toluene, xylene (single isomer or mixture of isomers), mesitylene, o-dichlorobenzene, nitrobenzene;
nitriles selected from a group comprising of acetonitrile, propionitrile or benzonitrile;
ethers selected from a group comprising of tetrahydrofuran, dimethoxyethane, diglyme, tetrahydropyran, methyltetrahydrofuran, butyltetrahydrofuran, p-dioxane;
amines selected from a group comprising of triethylamine, piperidine, pyridine, pyrrolidine, morpholine; amides selected from a group comprising of N,N-dimethylacetamide, N,N-dimethylformamide, N-methylpyrrolidinone, N-cyclohexylpyrrolidinone; aminoethers having formaulae R⁴R⁵NR⁶OR⁷NR⁸R⁹, R⁴OR⁶NR⁸R⁹, O(CH₂CH₂)₂NR⁴, R⁴R⁵NR⁶N(CH₂CH₂)₂O, R⁴R⁵NR⁶OR⁷N(CH₂CH₂)₂O, O(CH₂CH₂)₂NR⁴OR⁶N(CH₂CH₂)₂O; wherein R⁴⁻⁹ are independently selected from the group consisting of a linear or branched C1 to C₁₀ alkyl; and combinations thereof.

14. The system of claim 10, wherein the liquid metallic precursor has viscosity at ambient temperature between 1 cP and 10 cP.

15. The system of claim 10, wherein
the neutral (uncharged) metal compound is selected from the group consisting of dicobalthexacarbonyltert-butylacetylene [Co₂(CO)₆HC:::CC(CH₃)₃], (1-decyne) tetracobalt dodecacarbonyl (Co₄(CO)₁₂(C₈H₁₇C:::CH)), (1,6-Heptadiyne) tetracobalt dodecacarbonyl, (2,2,6-Trimethyl-3-heptyne) dicobalt hexacarbonyl,
(2,2-Dimethyl-3-octyne) dicobalt hexacarbonyl(CCTNBA), and Ru₃(CO)₉(PPh₂(CH₂)₃Si(OEt)₃)₃;
and
the solvent is selected from the group consisting of tetrahydrofuran, octane, hexane, toluene.

16. A vessel containing liquid metallic precursor comprising a neutral (uncharged) metal compound having a metal in zerovalent state and at least one neutral stabilizing ligand;
wherein
the metal is selected from the group consisting of Fe, Co, Ni, Ru, Ir, Rh, Pd, Pt, Cu, Ag, Au, Os, and combinations thereof;
the at least one neutral stabilizing ligand is selected from the group consisting of carbon monoxide (CO); nitric oxide (NO); dinitrogen (N₂); acetylene (C₂H₂); ethylene (C₂H₄); C₄-C₁₈ diene or C₄-C₁₈ cyclic diene; C₆-C₁₈ triene; C₈-C₁₈ tetraene; organo isocyanide RNC, wherein R is selected from the group consisting of C₁ to C₁₂ linear or branched hydrocarbyl or halocarbyl radical; organic nitrile RCN wherein R is selected from the group consisting of C₁ to C₁₂ hydrocarbyl or halocarbyl radical;
organophosphine PR′3 wherein R′ is selected from the group consisting of H, Cl, F, Br, and a C₁ to C₁₂ hydrocarbyl or halocarbyl radical; amine NRaRbRc, wherein Ra, Rb and Rc may be connected to each other and each is independently selected from H or a C₁ to C₁₂ hydrocarbyl or halocarbyl radical; organic ether R*OR**, wherein R* and R** can be connected to each other and each is selected independently from C₁ to C₁₂ hydrocarbyl or halocarbyl radicals; and terminal or internal alkyne with general formula R₁CCR₂, where R₁ and R₂ can be independently selected from the group consisting of H, C₁ to C₁₂ linear, branched, cyclic or aromatic halocarbyl or hydrocarbyl radical, silyl or organosilyl radical, stannyl or organostannyl radical, and combinations thereof;
the neutral (uncharged) metal compound is a liquid or a solid soluble at ambient temperature in a solvent selected from the group consisting of saturated linear, branched and cyclic hydrocarbons; or is a solid which melts at a temperature below a decomposition temperature;
the liquid metallic precursor has a viscosity at ambient temperature between 0.5 cP and 20 cP;
and
the vessel has a dip-tube extending beneath the surface of the liquid metallic precursor.

17. The vessel of claim 16, wherein
the terminal or internal alkyne is selected from the group consisting of propyne, 1-butyne, 3-methyl-1-butyne, 3,3-dimethyl-1-butyne, 1-pentyne, 1-hexyne, 1-decyne, cyclohexylacetylene, phenylacetylene, 2-butyne, 3-hexyne, 4,4-dimethyl-2-pentyne, 5,5-dimethyl-3-hexyne, 2,2,5,5-tetramethyl-3-hexyne, trimethysilylacetylene, phenyacetylene, diphenyl acetylene, trichlorosilylacetylene, trifluoromethylacetylene, cyclohexylacetylene, trimethylstannylacetylene, and combinations thereof;
the organophosphine is selected from the group consisting of phosphine (PH₃), phosphorus trichloride (PCl₃), phosphorus trifluoride (PF₃), trimethylphosphine (P(CH₃)₃), triethylphosphine (P(C₂H₅)₃), tributylphosphine (P(C₄H₉)₃), triphenylphosphine (P(C₆H₅)₃), tris(tolyl)phosphine (P(C₇H₇)₃), dimethylphosphinoethane ((CH₃)₂PCH₂CH₂P(CH₃)₂), diphenylphosphinoethane ((C₆H₅)₂PCH₂CH₂P(C₆H₅)₂), and combinations thereof;
the organic isocyanide is selected from the group consisting of methylisocyanide (CH₃NC), ethylisocyanide (C₂H₅NC), t-butylisocyanide ((CH₃)₃CNC), phenylisocyanide (C₆H₅NC), tolylisocyanide (C₇H₇NC), trifluoromethylisocyanide (F₃CNC), and combinations thereof;
the amine is selected from the group consisting of ammonia (NH₃), Trimethylamine ((CH₃)₃N), piperidine, ethylenediamine, pyridine, and combinations thereof;
the ether is selected from the group consisting of Examples of dimethylether (CH₃OCH₃), diethylether (C₂H₅OC₂H₅), methyltertbutylether (CH₃OC(CH₃)₃), tetrahydrofuran, furan, ethyleneglycoldimethylether (CH₃OCH₂CH₂OCH₃), diethyleneglycoldimethylether (CH₃OCH₂CH₂OCH₂CH₂OCH₃), and combinations thereof;
and
the organic nitrile is selected from the group consisting of acetonitrile (CH₃CN), propionitrile (C₂H₅CN), benzonitrile (C₆H₅CN), acrylonitrile (C₂H₃CN), and combinations thereof.

18. The vessel of claim 16, wherein the neutral (uncharged) metal compound is selected from the group consisting of
a. R¹Co₂(CO)₆, wherein R¹ is a linear or branched C₂ to C₁₀ alkyne, a linear or branched C₁ to C₁₀ alkoxy alkyne, a linear or branched C₁ to C₁₀ organoamino alkyne such as (tert-butylacetylene)dicobalt hexacarbonyl;
[Co₂(CO)₆HC:::CC(CH₃)₃];
b. R¹CoFe(CO)₇, wherein R¹ is a linear or branched C₂ to C₁₀ alkyne, a linear or branched C₁ to C₁₀ alkoxy alkyne, a linear or branched C₁ to C₁₀ organoamino alkyne;
c. R²CCo₃(CO)₉, wherein R² is selected from the group consisting of hydrogen, a linear or branched C₁ to C₁₀ alkyl, a linear or branched C₁ to C₁₀ alkoxy, Cl, Br, COOH, COOMe, COOEt;
d. R²CCo₂Mn(CO)₁₀, wherein R² is selected from the group consisting of hydrogen, a linear or branched C₁ to C₁₀ alkyl, a linear or branched C, to C₁₀ alkoxy, Cl, Br, COOH, COOMe, COOEt;
e. R³Co₄(CO)₁₂, wherein R³ is selected from a linear or branched C, to C₁₀ alkenylidene; and
f. R⁴Ru₃(CO)₁₁, wherein R⁴ is selected from the group consisting of a disubstituted alkyne (R^{#}CCR^{##}) wherein R^{#} and R^{##} can be selected independently from the group consisting of C₁ to C₁₂ linear, branched, cyclic or aromatic halocarbyl or hydrocarbyl radical, silyl or organosilyl radical, stannyl or organostannyl radical, and combinations thereof.

19. The vessel of claim 16, wherein the neutral (uncharged) metal compound is selected from the group consisting of dicobalthexacarbonyltert-butylacetylene [Co₂(CO)₆HC:::CC(CH₃)₃], (1-decyne) tetracobalt dodecacarbonyl (Co₄(CO)₁₂(C₈H₁₇C:::CH)), (1,6-Heptadiyne) tetracobalt dodecacarbonyl, (2,2,6-Trimethyl-3-heptyne) dicobalt hexacarbonyl, (2,2-Dimethyl-3-octyne) dicobalt hexacarbonyl, (2,2-Dimethyl-3-decyne) dicobalt hexacarbonyl(CCTNBA), (2,2-Dimethyl-3-heptyne) dicobalt hexacarbonyl, (tert-butylmethylacetylene)dicobalt hexacarbonyl (CCTMA), trirutheniumdodecacarbonyl, (ethylbenzene)(1,3-butadiene)Ruthenium, (isopropyl-4-methyl-Benzene)(1,3-butadiene)ruthenium, 1,3,5-cycloheptatrienedicarbonylruthenium, 1,3-cyclohexadienetricarbonylruthenium, 2,3-dimethyl-1,3-butadienetricarbonylruthenium, 2,4-hexadienetricarbonylruthenium, 1,3-pentadienetricarbonylruthenium, (benzene)(1,3-butadiene)ruthenium, (benzene)(2,3-Dimethyl-1,3-butadiene)ruthenium, Co₂Ru(CO)₁₁, HCoRu₃(CO)₁₃, Ru₃(CO)₉(PPh₂(CH₂)₃Si(OEt)₃)₃, bis(benzene)chromium, bis(cyclooctadiene)nickel, bis(tri-tert-butylphosphine)platinum, bis(tri-tert-butylphosphine)palladium, and combinations thereof.

20. The vessel of claim 16, wherein the solvent is selected from the group consisting of n-hexane, n-pentane, isomeric hexanes, octane, isooctane, decane, dodecane, heptane, cyclohexane, methylcyclohexane, ethylcyclohexane, decalin; aromatic solvent selected from a group comprising of benzene, toluene, xylene (single isomer or mixture of isomers), mesitylene, o-dichlorobenzene, nitrobenzene; nitriles selected from a group comprising of acetonitrile, propionitrile or benzonitrile; ethers selected from a group comprising of tetrahydrofuran, dimethoxyethane, diglyme, tetrahydropyran, methyltetrahydrofuran, butyltetrahydrofuran, p-dioxane; amines selected from a group comprising of triethylamine, piperidine, pyridine, pyrrolidine, morpholine; amides selected from a group comprising of N,N-dimethylacetamide, N,N-dimethylformamide, N-methylpyrrolidinone, N-cyclohexylpyrrolidinone; aminoethers having formaulae R⁴R⁵NR⁶OR⁷NR⁸R⁹, R⁴OR⁶NR⁸R⁹, O(CH₂CH₂)₂NR⁴, R⁴R⁵NR⁶N(CH₂CH₂)₂O, R⁴R⁵NR⁶OR⁷N(CH₂CH₂)₂O, O(CH₂CH₂)₂NR⁴OR⁶N(CH₂CH₂)₂O; wherein R⁴⁻⁹ are independently selected from the group consisting of a linear or branched C1 to C₁₀ alkyl; and combinations thereof.

21. The vessel of claim 16, wherein the liquid metallic precursor has viscosity at ambient temperature between 1 cP and 10 cP.

22. The vessel of claim 16, wherein
the neutral (uncharged) metal compound is selected from the group consisting of dicobalthexacarbonyltert-butylacetylene [Co₂(CO)₆HC:::CC(CH₃)₃], (1-decyne) tetracobalt dodecacarbonyl (Co₄(CO)₁₂(C₈H₁₇C:::CH)), (1,6-Heptadiyne) tetracobalt dodecacarbonyl, (2,2,6-Trimethyl-3-heptyne) dicobalt hexacarbonyl,
(2,2-Dimethyl-3-octyne) dicobalt hexacarbonyl(CCTNBA), and Ru₃(CO)₉(PPh₂(CH₂)₃Si(OEt)₃)₃;
and
the solvent is selected from the group consisting of tetrahydrofuran, octane, hexane, toluene.

23. A conductive metallic film deposited on a surface containing topography by using liquid metallic precursor comprising
a neutral (uncharged) metal compound selected from the group consisting of dicobalthexacarbonyltert-butylacetylene [Co₂(CO)₆HC:::CC(CH₃)₃], (1-decyne) tetracobalt dodecacarbonyl (Co₄(CO)₁₂(C₈H₁₇C:::CH)), (1,6-Heptadiyne) tetracobalt dodecacarbonyl, (2,2,6-Trimethyl-3-heptyne) dicobalt hexacarbonyl,
(2,2-Dimethyl-3-octyne) dicobalt hexacarbonyl(CCTNBA), and Ru₃(CO)₉(PPh₂(CH₂)₃Si(OEt)₃)₃;
and
a solvent selected from the group consisting of tetrahydrofuran, octane, hexane, toluene.

24. The conductive metallic film of claim 23 is deposited by spray coating, roll coating, spin coating, inkjet printing, dip-coating, and the combinations thereof.

25. The conductive metallic film of claim 23 has an electrical conductivity less or equal 1×10⁻⁴ Ωcm at ambient temperature.
